(19) United States
(12) Patent Application Publication
(10) Pub. No.: US 2003/0105434 A1
(43) Pub. Date: Jun. 5, 2003

(54) Protection cap assembly for syringes

(76) Inventor: Wei-Ting Chang, Nantou City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/996,889

(22) Filed: Nov. 30, 2001

(51) Int. Cl. A61M 5/00

(52) U.S. Cl. 604/198

(57) ABSTRACT

A syringe device includes a barrel having a needle cannular extending from an end plate on a first end thereof. A plunger is movably inserted into a second end of the barrel. A protection cap assembly comprises a first section and a second section which is mounted to a first end of the first section. A second end of the first section is movably mounted to a locking tip on the first end of the barrel. A stop member is connected to the first end of the first part and the needle cannula extends through an aperture in the first end of the first part. The stop member has a bounce force to contact the needle cannula so that when the first part is slid away from the locking tip, the needle cannula is received in the first part and the aperture is closed by the stop member.

### FIELD OF THE INVENTION

The present invention relates to a protection cap assembly for mounting on the locking tip of syringe and includes two sections and one of which has a stop member to avoid the needle cannula from penetrating therefrom after the syringe is used.

### BACKGROUND OF THE INVENTION

A conventional hypodermic syringe and generally includes a barrel with a locking tip on a first end thereof and a needle cannula is fixed to the locking tip. A plunger is movably received in the barrel so as to push the medicine in the barrel from the needle cannula. A protection cap is used to mount to the locking tip so as to receive the needle cannula in the cap. It is a serious concern that the needle cannula could hurt the medical personnel during removing the protection cap from the locking tip before proceeding syringing and/or re-mounting the protection cap to the locking tip after the syringing process is finished.

The present invention intends to provide a protection cap assembly by which the risk of injury of medical personnel can be reduced.

### SUMMARY OF THE INVENTION

In accordance with one aspect of the present invention, there is provided a syringe device which comprises a barrel having a locking tip extending from an end plate on a first end of the barrel and a plunger is movably inserted into a second end of the barrel. A needle cannula is fixed to the locking tip. A protection cap assembly comprises a first section and a second section which is mounted to a first end of the first section. A second end of the first section is an open end and movably mounted to the locking tip and a stop member is connected to the first end of the first part. An aperture is defined through the first end of the first section and the needle cannula extends through the aperture. The stop member has a bounce force to contact the needle cannula extending through the aperture.

The primary object of the present invention is to provide a syringe device wherein the needle cannula is received in a protection cap after being used.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the protection cap assembly of the present invention and a syringe device;

FIG. 2 is a cross sectional view to show the protection cap assembly and the syringe device before being used;

FIG. 3 shows that the stop member contacting the needle cannula;

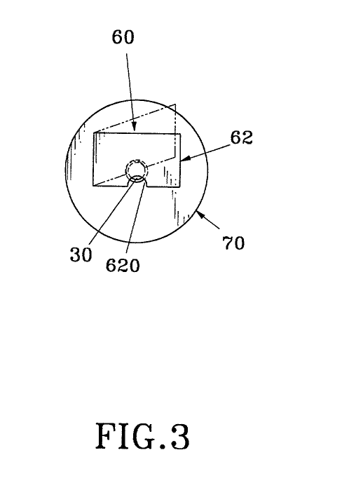

FIG. 4 shows that the needle cannula is received in the first section of the protection cap assembly;

FIG. 5 shows that the needle cannula is received in the first section of the protection cap assembly and a second part is to be connected to the first part;

FIGS. 6-9 are cross sectional view to respectively show various embodiments of the connections of the stop member and the first part;

FIG. 10 is a perspective view to show the first part and the stop member of the embodiment as shown in FIG. 9, and

FIG. 11 shows that two first parts are used to receive a longer needle cannula.

### DETAILED DESCRIPTION OF THE PREFERRED EMBODIMENT

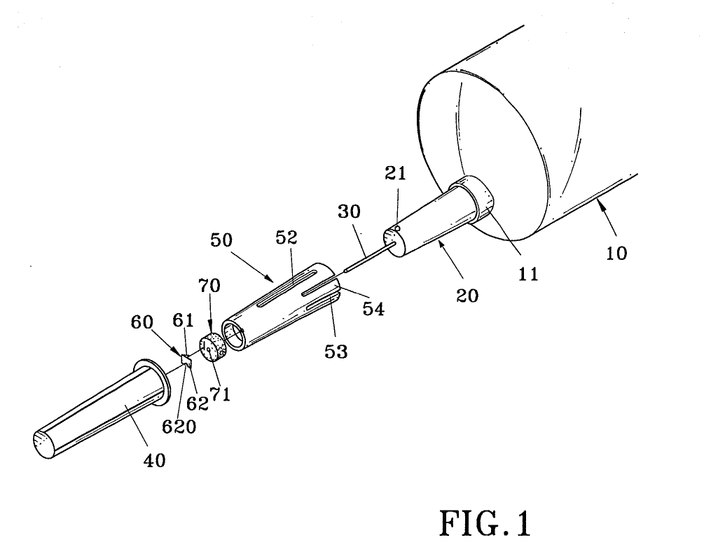

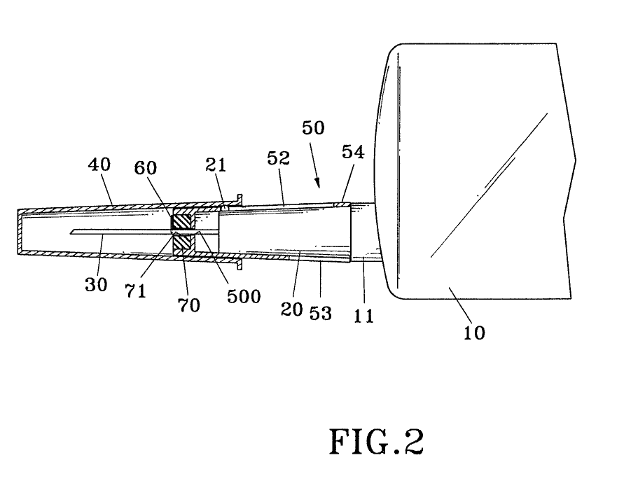

Referring to FIGS. 1 and 2, the syringe device of the present invention comprises a barrel 10 having a base 11 extending from an end plate on a first end of the barrel 10 and a locking tip 20 is connected to the base 11, A needle cannula 30 is fixed to the locking tip 20. A plunger 12 is movably inserted into the barrel 10 from a second of the barrel 10.

A protection cap assembly comprises a first section 50 and a second section 40 which is mounted to a first end of the first section 50. A second end of the first section 50 is an open end and has longitudinal recesses 53 defined in the second end of the first part 50 so as to form a flexible strip portion 54 which clamps the root of the locking tip 20. The first part 50 is movably mounted to the locking tip 20 and a slot 52 is defined through the first section 50. The locking tip 20 has a boss 21 extending radially from an outer periphery thereof and the boss 21 is engaged with the slot 52 so as to prevent the first part 50 from being separated from the locking tip 20.

An aperture 500 is defined through the first end of the first section 50 and a flexible member 70 is connected to the first end of the first part 50. A passage 71 is defined through the flexible member 70 and located in alignment with the aperture 500 in the first part 50. The needle cannula 30 extends through the aperture 500 and the passage 71.

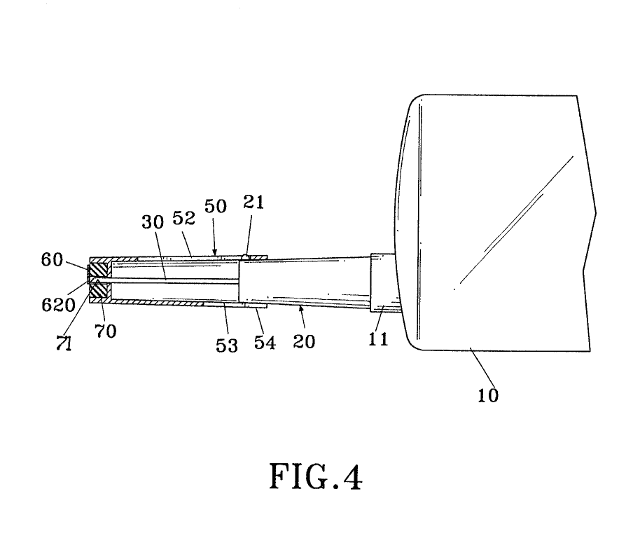

Referring to FIG. 4, a stop member 60 is an L-shaped member and includes an insertion 61 inserted in the flexible member 70, and a cover part 62 which is perpendicular to the insertion 61 and has semi-circular notch 620 defined in a side thereof. The needle cannula 30 is engaged with the notch 620. The cover part 62 has a bounce force and a tendency to cover the passage 71 up.

Before the syringe device is used, the needle cannula 30 is received in the second part 40 and is ready to use by removing the second part 50.

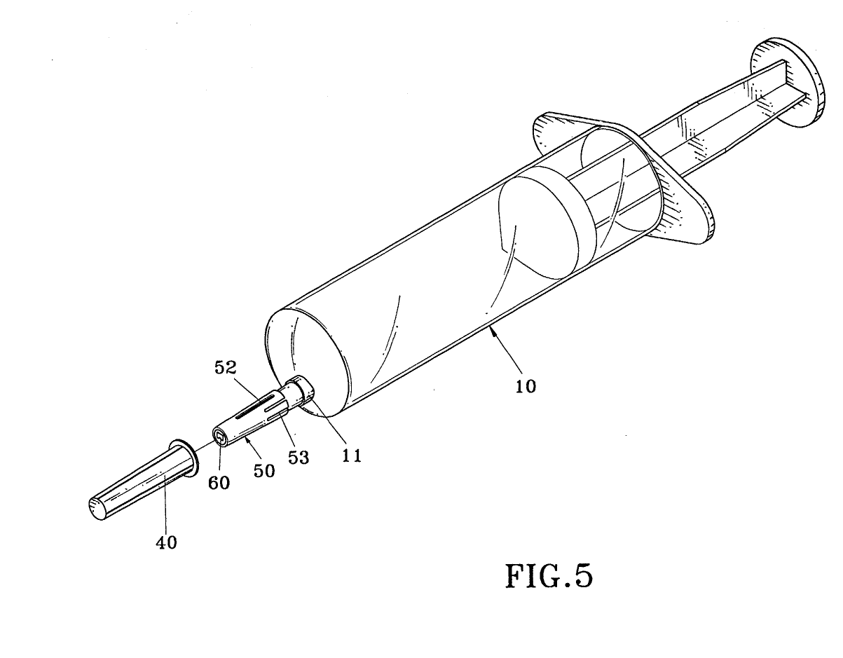

Referring to FIGS. 4 and 5, after the syringe device is used, the first part 50 is slid away from the locking tip 20 and to receive the needle cannula 30 in the first part 50. The stop member 60 is rotated when the needle cannula 30 is received in the first part 50 to cover the passage 71 up.

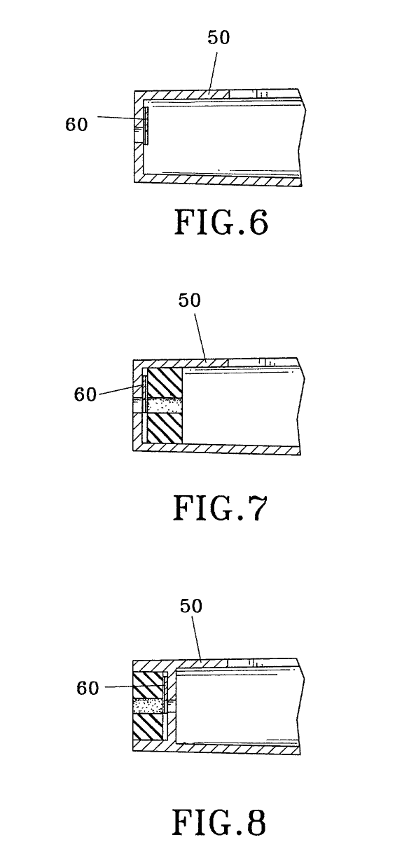

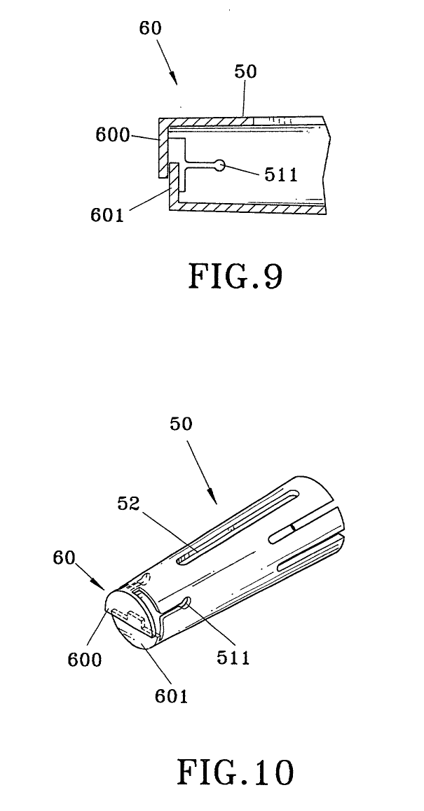

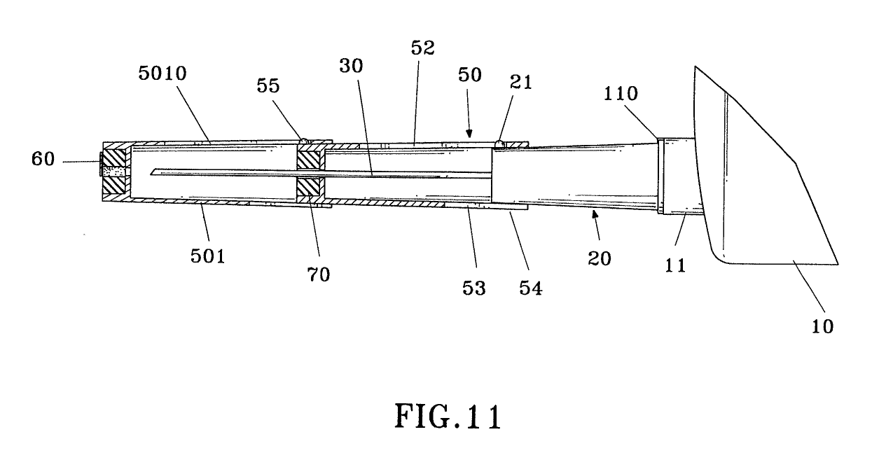

The stop member 60 can be connected to an inside of the first end of the first part 50 as shown in FIG. 6. FIG. 7 shows that the flexible member 70 and the stop member 60 are installed in the inside of the first end of the first part 50. FIG. 8 shows the stop member 60 is connected to the other end of the flexible member 70 as shown. FIGS. 9 and 10 show that two longitudinal grooves 511 are defined in the first end of the first part 50 so as to form two partitions 600, 601 separated by the grooves 511. The stop member 60 is composed by the two partitions 600, 601. Each of the two partitions 600, 601 has a plate perpendicular to a longitudinal axis of the first part 50 and the two plates of the two partitions 600, 601 can be pulled apart to allow the needle cannula 30 (not shown) to extend therebetween. FIG. 11 shows that two first parts 50, 501 are used to receive a longer needle cannula 30, and only the outer most first part 501 has the stop member 60. A hook 110 extends from the base 11 so as to hook the locking tip 20 which can be disengaged from the base 11 by releasing the hooking of the hook 110. The first part 50 connected to the hook 110 has a boss 55 and the first part 501 has a slot 5010 in which the boss 55 is received to limit the first part 501 from being separated from the first part 50.

While we have shown and described the embodiment in accordance with the present invention, it should be clear to those skilled in the art that further embodiments may be made without departing from the scope of the present invention.

What is claimed is:

1. A syringe device comprising:
a barrel having a locking tip extending from an end plate on a first end of said barrel, a plunger movably inserted into said barrel from a second of said barrel, a needle cannula fixed to said locking tip, and
a protection cap assembly comprising a first section and a second section which is mounted to a first end of said first section, a second end of said first section being an open end and movably mounted to said locking tip and a stop member connected to said first end of said first part, an aperture defined through said first end of said first section and said needle cannula extending through said aperture, said stop member having a bounce force to contact said needle cannula extending through said aperture.

2. The device as claimed in claim 1, wherein said needle cannula is received in said first section when said first section is pulled away from said locking tip.

3. The device as claimed in claim 1, wherein said locking tip has a boss extending radially from an outer periphery thereof and a slot defined through said first section, said boss engaged with said slot.

4. The device as claimed in claim l further comprising longitudinal recesses defined in said second end of said first part.

5. The device as claimed in claim 1 further comprising a flexible member connected to said first end of said first part and a passage defined through said flexible member and located in alignment with said aperture in said first part, said needle cannula extending through said passage, said stop member being an L-shaped member and including an insertion inserted in said flexible member and a cover part which has a side contacting said needle cannula.

6. The device as claimed in claim 5, wherein said side of said cover part has a semi-circular notch which engages with said needle cannula.

7. The device as claimed in claim 1 wherein two grooves defined longitudinally in said first end of said firs part so as to form two partitions separated by said grooves, each of said two partitions having a plate perpendicular to a longitudinal axis of said first part, said stop member being composed by said two it partitions, said two plates of said two partitions being pulled apart to allow said needle cannula to extend therebetween.
